(19) United States
(12) Patent Application Publication
(10) Pub. No.: US 2021/0244909 A1
(43) Pub. Date: Aug. 12, 2021

(54) NEUROFEEDBACK BASED SYSTEM AND METHOD FOR TRAINING MINDFULNESS

(71) Applicant: Roshan Narayan Sriram, Saratoga, CA (US)

(72) Inventor: Roshan Narayan Sriram, Saratoga, CA (US)

(21) Appl. No.: 17/242,272

(22) Filed: Apr. 27, 2021

Related U.S. Application Data
(63) Continuation-in-part of application No. 16/418,805, filed on May 21, 2019, now Pat. No. 11,013,449.

(51) Int. Cl. A61M 21/02; G06N 20/00; G06N 5/04; G09B 19/00

(52) U.S. Cl. CPC A61M 21/02; G06N 20/00; A61M 2021/0027; G09B 19/00; G06N 5/04

(57) ABSTRACT

A system and method for mind training that can help an individual to unlock his peak mental state. The system includes an EEG headgear configured to be worn over the head of a person. The EEG headgear can include a set of electrodes that can measure the electrical activity of the brain. A control unit can be operably coupled to the EEG headgear, such as to receive the electrical activity data from the EEG headgear. The control unit can include a machine learning model that can determine the mental state of an individual from the received electrical activity data. The system can also receive a desired mental state from the individual, wherein the machine learning model can determine the difference between the desired mental state and the current mental state and guide the individual to achieve the desired mental state.

### CROSS-REFERENCE TO RELATED APPLICATIONS

This application is a continuation-in-part of a U.S. Non-provisional patent application Ser. No. 16/418,805 filed May 21, 2019, which claims priority from a U.S. provisional patent application Ser. No. 62/592,560, filed Nov. 30, 2017, both of which are incorporated herein by reference in its entirety.

### FIELD OF INVENTION

The present invention relates to the fields of brainwave analysis, machine learning, and immersive virtual reality, and more particularly, the present invention relates to a system and method of training the mind to achieve mindfulness or the flow state.

### BACKGROUND

In today's world, the need for mental training is more than ever. Distraction has become a curse in the modern world. Be it professionals, students, sportsmen, or like, people cannot focus on their work, goals, and objectives. Lack of focus often leads to underperformance, anxiety, and mental disorders.

Mindfulness refers to a state of mind in which one is fully aware of what he is seeing and feeling at a moment. Flow is generally referred to as a state of mind in which a person becomes fully immersed in an activity. Meditation can help to unlock peak mental state achieving mindfulness and flow. However, meditation requires professional guidance and can be a slow and challenging process. Because of the complexities of the meditation process, people generally are less inclined to practice meditation or leave it in between. Moreover, the meditation is general and does not target any key mental state.

Thus, a desire is there for a system and method that can help a person in unlocking his or her peak mental state.

### SUMMARY OF THE INVENTION

The following presents a simplified summary of one or more embodiments of the present invention in order to provide a basic understanding of such embodiments. This summary is not an extensive overview of all contemplated embodiments and is intended to neither identify key or critical elements of all embodiments nor delineate the scope of any or all embodiments. Its sole purpose is to present some concepts of one or more embodiments in a simplified form as a prelude to the more detailed description that is presented later.

The principal object of the present invention is therefore directed to system and method for training mindfulness using neurofeedback mechanism.

It is another object of the present invention that the system and method could train individuals to achieve peak mental states and unlock their full potential.

It is still another object of the present invention that system and method enable businesses & organizations to empower employees to achieve mental well-being.

It is yet another object of the present invention that system and method can guide performers into peak mind/body states, such as the “flow state”, through personalized, closed loop, fully immersive experiences.

It is a further object of the present invention that the system and method provide for enriching the quality of life.

It is still a further object of the present invention that the system and method provide for mental resilience to be better equipped to handle real-life circumstances and such as an individual can maintain the flow state while engaging in real-world activities.

Disclosed is a system and method for mind training that can help an individual to unlock his peak mental state. The disclosed system and method can help an individual to achieve mindfulness and flow state. The disclosed system can include an EEG headgear configured to be worn over the head of a person. The EEG headgear can include a set of electrodes that can measure the electrical activity of the brain. A control unit can be operably coupled to the EEG headgear, such as to receive the electrical activity data from the EEG headgear. The control unit can include a machine learning model that can determine the mental state of an individual from the received electrical activity data. The disclosed system can also receive a desired mental state from the individual, wherein the machine learning model can determine the difference between the desired mental state and the current mental state and guide the individual to achieve the desired mental state.

In one aspect, the machine learning model can be trained using the dataset that includes mental state data of a range of people evaluating the change in their mental states under different conditions. The conditions can include relaxed, focused, stressed, happy, sad, energetic, etc. The people can include spiritual persons and the change in the mental state before, during, and after the meditation can be used to train the machine learning models. Similarly, the mental state of people who excel in their fields can also be used to train the disclosed machine learning model.

In one aspect, the disclosed system can also include a speaker to broadcast voice and audio stimuli and feedback. The control unit can both visually and through voice guide an individual in achieving the desired mental state. Moreover, the disclosed system can determine the sounds and voices that positively affect the mind. Such a voice that positively impacts the mental state can be used to guide the user.

In one aspect, the disclosed system provides a dynamic flame to manipulate the mental state of a user.

In one aspect, the disclosed system and method can help like-minded people to find each other and socialize, such as on the social network. The disclosed system can suggest friends based on the mental state of a person.

In one aspect, the disclosed system may act as a coach that can guide a person in day-to-day activities to maintain focus and enhance performance.

These and other objects and advantages of the embodiments herein and the summary will become readily apparent from the following detailed description taken in conjunction with the accompanying drawings.

### BRIEF DESCRIPTION OF THE DRAWINGS

The accompanying figures, which are incorporated herein, form part of the specification and illustrate embodiments of the present invention. Together with the description, the figures further explain the principles of the present invention and to enable a person skilled in the relevant arts to make and use the invention.

FIG. 1 shows an EEG headgear, VR headset, and an audio emitter of the disclosed system worn by a user, according to an exemplary embodiment of the present invention.

FIG. 2 shows an exemplary embodiment of the dynamic environment that can be presented to a user, according to an exemplary embodiment of the present invention.

FIG. 3 shows the properties of a dynamic flame, according to an exemplary embodiment of the present invention.

FIG. 4 shows a method of mind training, according to an exemplary embodiment of the present invention.

FIG. 5 is a flow chart showing the steps of mind training, according to an exemplary embodiment of the present invention.

FIG. 6 is an accuracy plot of training the machine learning model, according to an exemplary embodiment of the present invention.

FIG. 7 is a scatter plot of features learned by the machine learning model visualized in two dimensions, according to an exemplary embodiment of the present invention.

FIG. 8 is a chart showing the Power Spectrum Density (PSD) of the meditative state and the normal state, according to an exemplary embodiment of the present invention.

### DETAILED DESCRIPTION

Subject matter will now be described more fully hereinafter with reference to the accompanying drawings, which form a part hereof, and which show, by way of illustration, specific exemplary embodiments. Subject matter may, however, be embodied in a variety of different forms and, therefore, covered or claimed subject matter is intended to be construed as not being limited to any exemplary embodiments set forth herein; exemplary embodiments are provided merely to be illustrative. Likewise, a reasonably broad scope for claimed or covered subject matter is intended. Among other things, for example, the subject matter may be embodied as methods, devices, components, or systems. The following detailed description is, therefore, not intended to be taken in a limiting sense.

The word “exemplary” is used herein to mean “serving as an example, instance, or illustration.” Any embodiment described herein as “exemplary” is not necessarily to be construed as preferred or advantageous over other embodiments. Likewise, the term “embodiments of the present invention” does not require that all embodiments of the invention include the discussed feature, advantage, or mode of operation.

The terminology used herein is for the purpose of describing particular embodiments only and is not intended to be limiting of embodiments of the invention. As used herein, the singular forms “a”, “an” and “the” are intended to include the plural forms as well, unless the context clearly indicates otherwise. It will be further understood that the terms “comprises”, “comprising,”, “includes” and/or “including”, when used herein, specify the presence of stated features, integers, steps, operations, elements, and/or components, but do not preclude the presence or addition of one or more other features, integers, steps, operations, elements, components, and/or groups thereof.

The following detailed description includes the best currently contemplated mode or modes of carrying out exemplary embodiments of the invention. The description is not to be taken in a limiting sense but is made merely for the purpose of illustrating the general principles of the invention, since the scope of the invention will be best defined by the allowed claims of any resulting patent.

Presented herein are a neurofeedback system and method for monitoring, analyzing, training, and inducing peak mental states. An EEG headband is worn by the user to measure the user's mental state. A machine learning model can interpret the measured signals into the mental state of the user, such as relaxed, focused, stressed, happy, sad, energetic, etc. The system can obtain a desired mental state of the user, such as relaxed, energized, etc. The system can measure the difference between the desired mental state and the measured mental state of the user and provide audio and visual guidance to the user indicating how far the user is from the desired mental state. Furthermore, the system can provide audio and visual feedback to help or challenge the user in reaching the desired mental state. The visual and audio guidance can be provided using virtual reality and/or an augmented reality product.

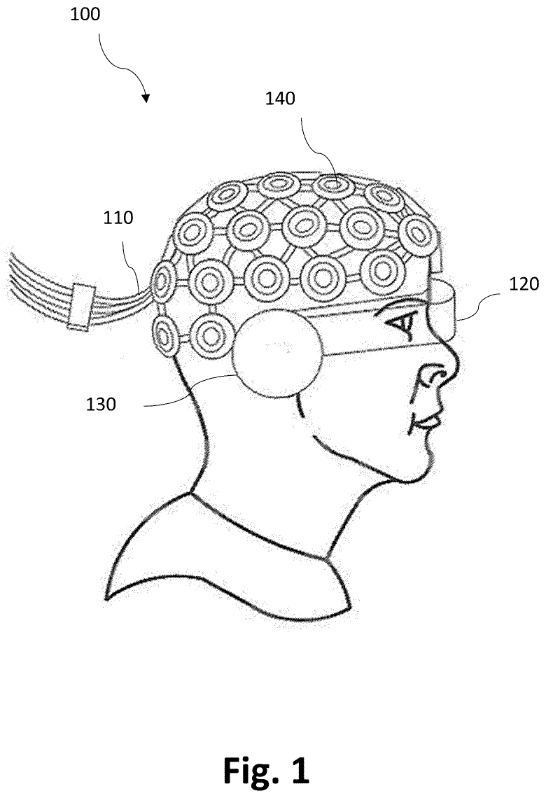

Referring to FIG. 1 which shows an exemplary embodiment of the disclosed system 100 having an EEG headgear 110 worn by a user. FIG. 1 also shows a VR display 120 and an audio emitter 130. The headgear 110 can include several brainwave sensors 140 that can measure brainwave signals, such as EEG electrodes and eye movement trackers can be used. Other sensors, such as Galvanic Skin Response, Electrocardiograms, Seismocardiogram, and Ballistocardiogram sensors can also be worn by the user. System 100 can also include a control unit (not shown) that can be connected to the EEG headgear, VR headset, and audio emitter. The control unit can include a pre-trained machine learning model that can map the electrical activity of the brain from the electrical signals received from the brain sensors. The pre-trained machine learning model can analyze the brainwave data to determine the mental state of the user, such as relaxed, focused, stressed, happy, sad, etc. The control unit can also receive an end objective of the user, such as an athletic wish to improve their athletic performance and an employee wish to improve their work performance. Based on the received objective by the control unit, the control unit can determine a desired mental state associated with the objective of the user. The desired mental state is generally based on three principles, also referred herein as the 3C's of mindfulness i.e., concentration, clarity, and composure. Concentration refers to the ability to focus on what the user chooses at a given time. The concentration can empower the athlete to sustain and shift their attention at will. Clarity is the ability to track and explore one's senses in real-time. Better clarity empowers an athlete to be highly aware of what is happening within the body and around. Composure can be an ability to allow one's sensory experience to come and go without push and pull. The composure can empower an athlete to be resilient and overcome adverse situations.

The machine learning model can be trained to encapsulate all the above three mind states into one and unique metric. The machine learning model can determine the difference between the current mental state and the desired mental state of a user and tracks the performance of the user. The disclosed system aims to enhance one's mental resilience to be better equipped to handle real-life circumstances and to maintain one's “flow state” while interacting in real-life situations.

The sensors 110 can include multiple electrodes 140 (only one labeled for brevity) placed along the scalp of the user and continually detecting electrical activity of the brain. The waveform so produced is the brainwave signal, which can have varying frequencies depending on the user's mental state. The gamma brainwave signal has frequencies in the 30 to 100 Hz range and can occur when the user is in a heightened sense of consciousness, bliss, and intellectual acuity. The beta brainwave signal has frequencies in the 14 to 30 Hz range and can occur when the user is awake and mentally active. The alpha brainwave signal has frequencies in the 8 to 14 Hz range and can be generated when the user is awake and resting. The theta brainwave signal has frequencies in the 3.5 to 8 Hz range and can be generated when the user is sleeping. The delta brainwave has frequencies less than 3.5 Hz and can be generated when the user is in deep sleep.

In a meditative state, increased alpha brainwave signals and gamma brainwave signals occur. For example, when the user closes his\her eyes, there is an increase in the alpha brainwave signal. Further, in deep meditation, the gamma brainwave signal can be detected most commonly around 40 Hz. To encourage the user to reach a desired mental state, such as relaxation meditation, visual and audio guidance can be provided to the user and also including neurofeedback indicating the closeness of the user to the desired mental state. Further, the user can be incentivized to practice reaching and maintaining the desired mental state by tracking the user's past performance in reaching and maintaining the desired mental state.

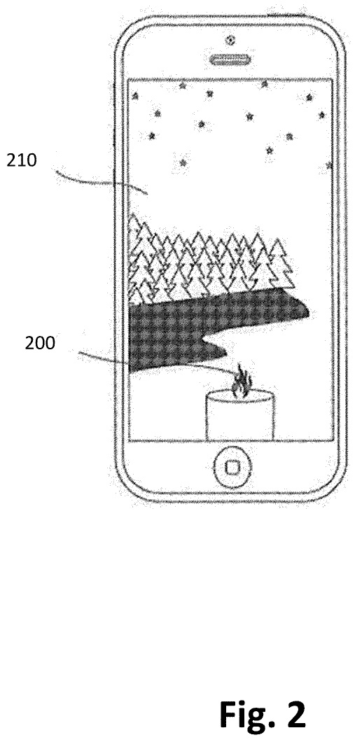

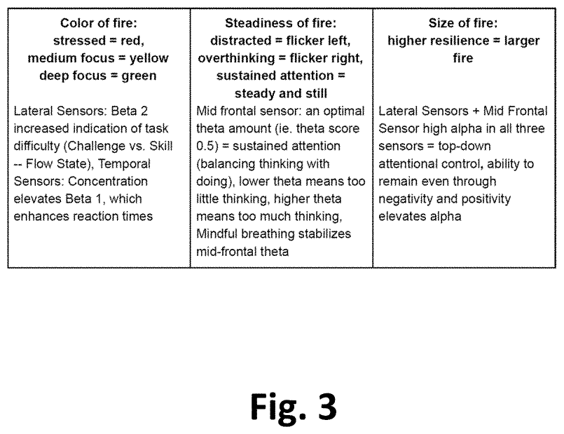

FIG. 2 shows an exemplary embodiment of the visual guidance provided to a user using the disclosed immersive VR headset, according to one embodiment. Driving users into peak mental state requires carefully designed immersive environments that enunciate mental state, stimuli, and feedback given to the user. The flame object 200 represents carefully chosen brain stimuli and a neurofeedback symbol that can help a user meditate and unlock his peak mental states. Flame is a dynamic and multi-dimensional continuous object capable of depicting a holistic visualization of the user's mental state. Moreover, as shown in FIG. 2, a dynamic environment 210 that can represent the current mental state of the user presented through the disclosed immersive VR headset combined with the audio. The dynamic environment can include clouds, wind, and fire. The natural landscape is generally soothing to a user's mind and the dynamic environment based on the natural landscape including fire, wind, and clouds can allow the user to quickly progress to higher mental states reaching closer to the desired mental state. Flame is generally not considered soothing, but energetic. Fire represents a symbolic tie-in of the Olympic Fire. Keeping the fire lit through adverse circumstances signifies the victory of positive over negative, as embodied in the present invention where positive mind states keep the fire lit whereas negative mind states put the fire dull. The soothing landscape and the energetic fire help with the 3C's of Mindfulness. The three mind states that unlock peak meditative states can signify a specific aspect of the fire (i.e., color a concentration, steadiness a calmness, size a resilience) as shown in FIG. 3.

The training sessions for a user can be further customized according to the profile of the user. The profile can include details of any mental disorders, phobias, work, hobbies, likes, and dislikes of a user. The machine learning model can in near real-time track the progress of a user. The machine learning model based on the progress of the user can personalize the training sessions. This leap in understanding unlocks an unprecedented degree of personalization, recommendations, curations, etc. of content that is unique and specific. The user can be exposed to a realistic environment for both training and tracking the progress of the mind training. The disclosed system can provide a safe environment for exposing the user to his phobias. The virtual immersive environment works as a safe space for patients to confront their phobias. The personalized training program can help develop anxiety management skills over a series of sessions. The training sessions can provide realistic environments based on different phobias, such as acrophobia, herpetophobia, Ochlophobia, Hydrophobia, Claustrophobia, and like.

The disclosed system can provide a leap in terms of Virtual Reality solutions for Cognitive Behavioral Therapy. The machine learning model through the immersive virtual reality environment can provide for treating Anxiety Disorders like Panic Disorders, PTSD, Social Anxiety, Phobia Treatment (Acrophobia, Hydrophobia, Arachnophobia, Agoraphobia, Ochlophobia, and others).

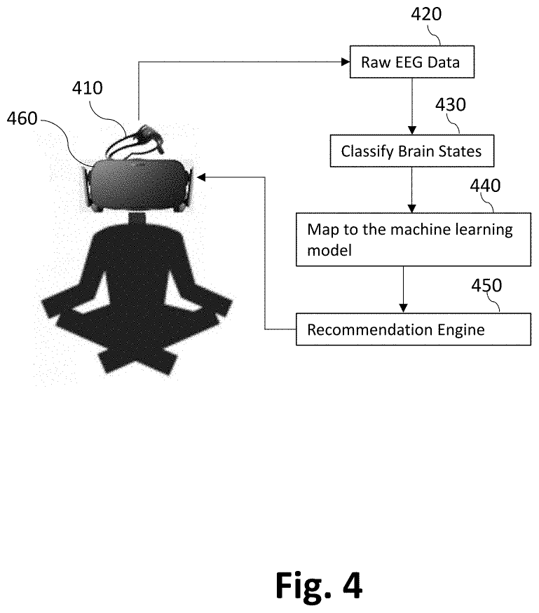

The disclosed EEG headgear 410 using different sensors including eye motion tracking can help in mapping the brain activity. Referring to FIG. 4, the processor of the control unit can transform the measured brainwave signals into the mental state of the user. The raw EEG data can be received by the disclosed control unit, at step 420, which can classify the brain state into cognitive, emotional, and Epistemic brain states, at step 430. The cognitive state can be associate with focus and the disclosed system can measure the current level of focus using the brainwave signal. The emotional mental state can be associated with relaxation and can be used to judge calmness and composure. An epistemic state of mind can reflect the interest and can be used to judge the level of interest.

The classified mental states can be mapped to the machine learning model, at step 440. Machine learning can include algorithms including never-ending attention learner (N.E.A.L) for cognitive profiling (attention and learning); never-ending emotional learner (N.E.E.L) profiling the memory (Likes, Dislikes); and never-ending human learner (N.E.H.L) for personality profiling (Beliefs and values). The disclosed machine learning model can also include a recommendation engine to provide near-real-time feedback, rewards, and analytical report of the progress of the user, at step 450. The neuronal feedback can be the current mental state of the user and shows whether the user is getting distracted from the predefined path. The neural feedback can be combined with measures including audio and video stimuli to bring the user's mental state back to the predefined path, at step 460. For example, focusing can make the flame brighter orange, whereas being distracted can make the flame emerald green or sapphire blue. If the user is too stressed, the weather can be cloudy and raining, whereas if the user is calm, the weather can be peaceful and serene. To have the foreground and background interact, the brighter flames can be more resistant to the weather. This is particularly advantageous to train the composure aspect of mindfulness. Consequently, if a person is both distracted and stressed, the flame can go out and the session can end, or the user can be penalized in terms of the overall score for the session. If the user is focused and stressed, but the user manages to calm down, the user can progress in the session. If the user is relaxed but distracted, the flame can become duller and duller without going out, giving the user the stimuli to refocus their attention on the tip of the flame. In another example, the more focused the user is, the bigger the flame becomes, and the calmer the user is the steadier the flame becomes. Also, the system can provide audio stimuli. If the user is distracted, the audio stimuli can include the sound of the wind, and if the user is calmer, the sound of the wind can subside. As the user's mental state approaches the desired mental state, the visual and/or audio feedback can indicate the proximity to the desired mental state by steadying the flame and/or reducing the amplitude of the sound of the wind, thus rewarding the user.

Another element of the waves hitting the shores and the sound of waves can also be included in the dynamic environment. It is obvious that the inclusion and exclusion of elements in the dynamic environment can be customized based on the profile of the user and the neuronal feedback in response to the dynamic environment. Moreover, the dynamic environment to which a user is exposed may not be the same in all the training sessions but can be customized based on the neurofeedback by including or substituting the elements like waves hitting the shores. Training sessions can include both the customized dynamic environment, realistic environment, and flow state exercises.

The waves can represent a distracted current mental state, wherein more is the distraction more is the waves hitting the shore and increase audio of winds and waves hitting off the shores. As the user's brainwave signal gets closer to the desired brainwave signal, the waves subside, and the dynamic environment becomes calmer while flame becomes steady and growing.

The weather can be adjusted based on the user's level of relaxation. For example, if the user is too stressed, the weather can take on the quality of a storm and the waves can be harsh. However, as the user starts to relax, the weather can start to calm down and waves can become smooth. The system can establish a maximum intensity threshold for the negative stimulus to prevent the feedback from overwhelming the user. Moreover, the level of focus and relaxation, i.e., the desired mental state, needed to achieve the predefined objectives can be dynamically adjusted based on the user's mindfulness proficiency, i.e., skill level.

It is obvious that different users may differently respond to the training. Some users may reach the touchpoints in the journey to the predefined objective quicker than other users. Thus, the disclosed system can customize the training sessions based on the ability of the user and prevents any training fatigue and stress.

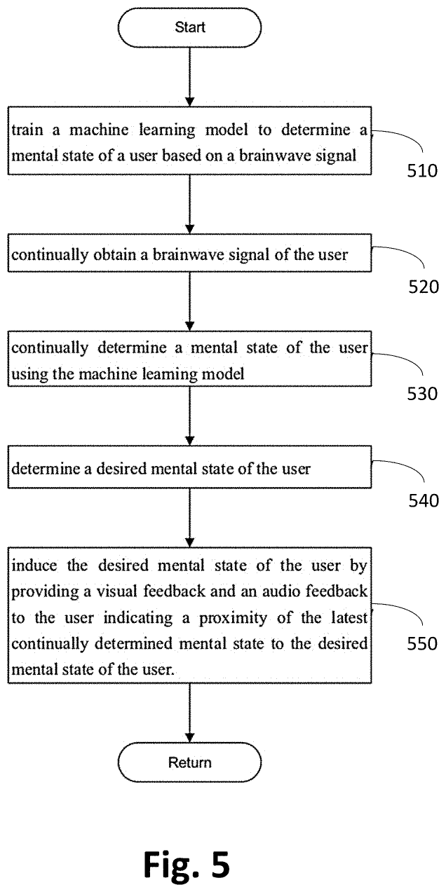

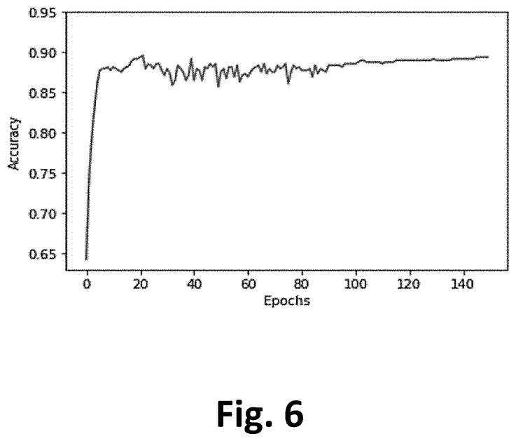

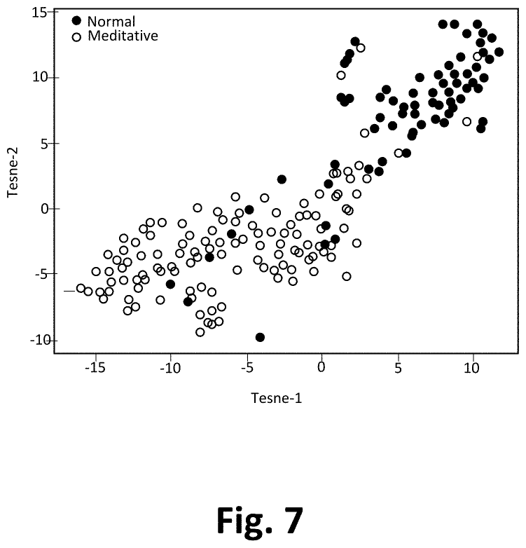

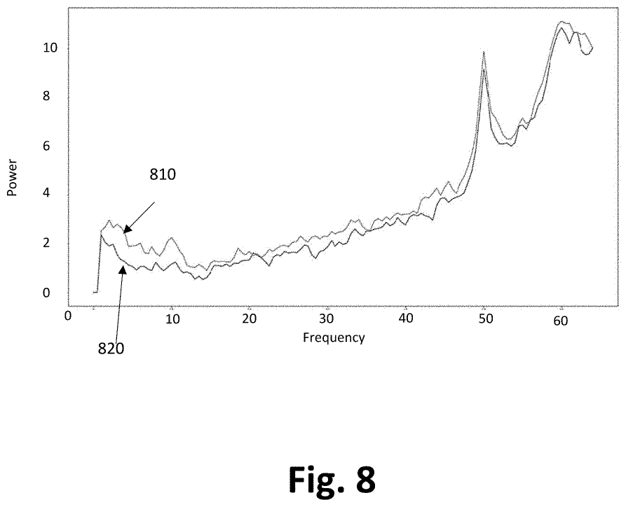

Referring to FIG. 5 which is a flow chart showing the method of mind training according to an exemplary embodiment of the present invention. First, the machine learning model can be trained at step 510. The machine learning model can be a pre-trained machine learning model that can be trained using a dataset prepared by studying the mental state of spiritual leaders, ascetic persons, and/or monks. In one case, the subjects can be engaged in three sessions each 10 minutes long. Session I may include studying the mental state in a normal state. Session II may include studying the meditative state and session III may include studying the meditative state with disturbance after every 30 seconds. The dataset was first manually labeled with a total of three labels i.e., the normal state, the meditative state, and the disturbances. The starting time of the disturbance was taken from the transcripts, however, how long it took for the monk to go back into the meditative state was not known. So, for experiments, these disturbances were assumed to have disruptive effects for three seconds. Once the labeling is done, the data from all the monks were combined and segmented into one-second windows. The data is also shuffled so that the Deep Learning framework is not affected by the variance in the signals of each participant i.e., the algorithm does not only learn features participant-specific features. A deep convolutional network is trained with the aim of classifying every one-second segment into either a meditative or normal state. For now, the disturbances are considered as a normal state or the non-meditative state. Train, validation, and test split were roughly 0.7, 0.1, and 0.2. Classification accuracy achieved by the network on the test set was 90%. FIG. 6 shows the accuracy achieved by the deep learning framework over the training session. FIG. 6 is the accuracy plot after every epoch of training. FIG. 7 shows the difference between features learned for meditative and the normal state visualized in a two-dimensional plot. From the chart, it is clear that the model was able to separate the two states since the same states are clustered together and are separate from each other. Analyzing the Power Spectrum Density (PSD) as shown in FIG. 8 where line 810 shows PSD of meditative state and the line 820 show PSD of a normal state, it can be observed that most of the channels have quite a similar power spectrum except for T7, P7, F8 and AF4 where Delta and Theta bands are more profound for a meditative state. So average power/hertz is more in a meditative state as compared to the normal state.

The pre-trained machine learning model can determine a mental state of a user based on a brainwave signal. The machine learning model can continuously obtain the brainwave signal in near real-time in a training session and real-world activities, at step 520. The brainwave signal data can be used to determine the current mental state of the user, at step 530 using the machine learning model. The disclosed system can also obtain the desired mental state from the stated objectives and/or goals of the user, at step 540. The disclosed system can train the mind of the user to achieve the desired mental state by audio and visual stimuli and tracking the progress of the user in near real-time. The disclosed system can provide personalized training sessions for the user that can include audio and video stimuli. The training data can include the above-described dynamic environment, realistic environment based on the profile of the user, and flow state training exercises. This includes real-time voice guidance from mental wellness coaches, audio cues (both mindfulness and music), and visual stimuli that are adjusted in real-time based on the mental state the user is in at that moment in time, at step 550.

While the foregoing written description of the invention enables one of ordinary skill to make and use what is considered presently to be the best mode thereof, those of ordinary skill will understand and appreciate the existence of variations, combinations, and equivalents of the specific embodiment, method, and examples herein. The invention should therefore not be limited by the above-described embodiment, method, and examples, but by all embodiments and methods within the scope and spirit of the invention as claimed.

What is claimed is:

1. A method for guiding a user to achieve a desired mental state, the method comprising the steps of:
receiving, from an EEG headset worn by a user, by a control unit, in near real time, an electrical activity of a brain of the user, the control unit comprises a machine learning model;
determining, by the control unit, one or more features of a current mental state of the user from the electrical activity;
determining, by the control unit, a desired mental state based on an input from the user;
presenting, by the control unit, through an immersive virtual reality headset, an interactive dynamic environment to stimulate the brain for achieving the desired mental state, the interactive dynamic environment comprises a flame, an immersive virtual reality headset also comprises at least one speaker;
manipulating, the interactive dynamic environment in near real time, based on the one or more features of the current mental state of the user and a difference between the desired mental state and the one or more features of the current mental state of the user.

2. The method according to claim 1, wherein the method further comprises the steps of training the machine learning model, wherein the step of training the machine learning model comprises:
determining a first mental state of a plurality of subjects during non-meditation activities;
determine a second mental state of the plurality of subjects during meditation; and
determining a third mental state of the plurality of subject during meditation with periodic distractions.

3. The method according to claim 1, wherein the method further comprises the steps of:
receiving, by the control unit, a user profile of the user, the user profile comprises user's interests, one or more phobias of the user, and a work environment of the user;
generating, by the control unit, a realistic environment based on the one or more phobias of the user;
presenting, by the control unit, the realistic environment through the immersive virtual reality headset to the user; and
upon presenting, determining, by the control unit, the one or more features of the current mental state of the user.

4. The method according to claim 1, wherein the flame is configured to vary in color, intensity, and flickering based on the one or more features of the current mental state.

5. The method according to claim 4, wherein the interactive dynamic environment further comprises clouds and sound of wind.

6. The method according to claim 4, wherein the one or more features of the current mental state are based on current levels of concentration, calmness, and composure, wherein increasing levels of the concentration on the flame results in increase in the size of the flame.

7. The method according to claim, 6, wherein the clouds and winds can vary in intensity based on the current levels of the calmness and the composure.

8. The method according to claim, 6, wherein the intensity of clouds and winds are inversely proportional to the current levels of the calmness and the composure.

9. The method according to claim 1, wherein the interactive dynamic environment comprises a background and a foreground, wherein the background configured to manipulate the foreground, the foreground configured to resist the background.

10. The method according to claim 9, wherein the foreground comprises the flame, the background comprises a landscape, the landscape comprises clouds and wind.

11. The method according to claim 10, wherein the landscape further comprises waves hitting a shore.
